(19) United States
(12) Patent Application Publication
(10) Pub. No.: US 2013/0251096 A1
(43) Pub. Date: Sep. 26, 2013

(54) X-RAY COMPUTED TOMOGRAPHY APPARATUS

(71) Applicants: Toshiba Medical Systems Corporation, Otawara-shi (JP); Kabushiki Kaisha Toshiba, Minato-ku (JP)

(72) Inventor: Manabu HIRAOKA, Nasushiobara-shi (JP)

(73) Assignees: Toshiba Medical Systems Corporation, Otawara-shi (JP); Kabushiki Kaisha Toshiba, Minato-ku (JP)

(21) Appl. No.: 13/892,622

(22) Filed: May 13, 2013

Related U.S. Application Data
(63) Continuation of application No. PCT/JP2012/077604, filed on Oct. 25, 2012.

(30) Foreign Application Priority Data
Oct. 25, 2011 (JP) 2011-234037

(51) Int. Cl. A61B 6/03

(52) U.S. Cl. CPC A61B 6/032
USPC 378/8

(57) ABSTRACT

According to one embodiment, an X-ray CT apparatus includes units. The unit calculates an arrhythmia index. The unit selects a normal mode or an arrhythmia mode based on the arrhythmia index. In the normal mode, after the elapse of a predetermined delay time from the characteristic wave, projection data is collected along with X-ray generation. Upon detecting an arrhythmia, the X-ray generation is temporarily stopped. In the arrhythmia mode, after the elapse of the predetermined delay time, projection data is collected along with X-ray generation. Upon detecting an arrhythmia, an arrhythmia scanning procedure corresponding to an arrhythmia type is temporarily executed, and projection data is collected along with the X-ray generation.

### CROSS REFERENCE TO RELATED APPLICATIONS

This application is a Continuation Application of PCT Application No. PCT/JP2012/077604, filed Oct. 25, 2012 and based upon and claiming the benefit of priority from Japanese Patent Application No. 2011-234037, filed Oct. 25, 2011, the entire contents of all of which are incorporated herein by reference.

### FIELD

Embodiments described herein relate generally to an X-ray computed tomography apparatus.

### BACKGROUND

ECG-gated scan enables to collect data in a desired cardiac phase such as a middiastolic phase by starting data when a predetermined standby time has elapsed from an R wave that modifies the X-ray intensity (increases/decreases the X-ray tube current or turns on/off the X-ray output) in synchronism with the signal from an electrocardiograph. The ECG-gated scan is often used for cardiac CT.

However, if the heart rate (heartbeat cycle) is unstable due to an arrhythmia or the like, it is impossible to collect data in a desired cardiac phase. In this case, collected data is wasted. In addition, if retry of data collection is repeated, the scanning time prolongs, and reinjection of the contrast medium may be needed.

As a method of avoiding wasteful data collection and reducing unnecessary dose, a situation in which the heart rate (heartbeat cycle) largely varies is determined as an arrhythmia, X-ray generation is stopped, and the scanning is resumed from the next heartbeat. However, prolongation of the scanning time and reinjection of the contrast medium are unavoidable even by this control.

### CITATION LIST

### Patent Literatures

Patent Literature 1: Jpn. Pat. Appln. KOKAI Publication No. 2005-066042

### BRIEF DESCRIPTION OF THE DRAWINGS

FIG. 1 is a block diagram showing the arrangement of an X-ray computed tomography apparatus according to an embodiment of the present invention.

FIG. 2 is a flowchart showing an operation of the embodiment.

FIG. 3 is an explanatory view of heartbeat cycle classification by an arrhythmia type determination unit shown in FIG. 1.

FIG. 4 is a graph showing the frequency distribution of heartbeat cycles by the arrhythmia type determination unit shown in FIG. 1 together with the classification.

FIG. 5 is a view showing an example of an scanning plan setting screen by an scanning plan support unit shown in FIG. 1.

FIG. 6 is a view showing another example of the scanning plan setting screen by the scanning plan support unit shown in FIG. 1.

FIG. 7 is a view showing still another example of the scanning plan setting screen by the scanning plan support unit shown in FIG. 1.

FIG. 8 is a view showing an example of an scanning procedure corresponding to a ventricular compensatory type by a mode selection unit shown in FIG. 1.

FIG. 9 is a view showing an example of an scanning procedure corresponding to an extrasystole (compensatory) type by the mode selection unit shown in FIG. 1.

FIG. 10 is a view showing an example of an scanning procedure corresponding to an extrasystole (interpolated) type by the mode selection unit shown in FIG. 1.

FIG. 11 is a flowchart showing another operation of the embodiment.

FIG. 12 is a view showing an example of an operation selection screen displayed in an operation selection screen display step shown in FIG. 11.

FIG. 13 is a flowchart showing the operation procedure of performing arrhythmia detection and arrhythmia type specification using a cardiogram according to the embodiment.

### DETAILED DESCRIPTION

In general, according to one embodiment, an X-ray computed tomography apparatus includes an arrhythmia index calculation unit and a mode selection unit in addition to an X-ray tube, a high-voltage generation unit, an X-ray detector, and a data collection unit. The arrhythmia index calculation unit calculates an arrhythmia index representing the degree of arrhythmia using the biological information of an object. The mode selection unit selects a normal imaging mode (normal scanning mode) or an arrhythmia imaging mode (arrhythmia scanning mode) in advance based on the arrhythmia index. In the normal imaging mode, after the elapse of a predetermined delay time from the characteristic wave of the heartbeat waveform of the object, projection data is collected along with X-ray generation. Upon detecting an arrhythmia, the X-ray generation is temporarily stopped. In the arrhythmia imaging mode, after the elapse of a predetermined delay time from the characteristic wave, projection data is collected along with X-ray generation. Upon detecting an arrhythmia, an arrhythmia scanning procedure corresponding to an arrhythmia type is temporarily executed, and projection data is collected along with the X-ray generation.

An embodiment of the present invention will now be described with reference to the accompanying drawings. Note that there exist various types of X-ray computed tomography apparatuses including a rotate/rotate type that integrally rotates an X-ray tube and a radiation detector around an object, and a stationary/rotate type that arrays a number of detection elements in a ring shape and rotates only an X-ray tube around an object. The present invention is applicable to any type. The rotate/rotate type that is the current mainstream will be explained here. To reconstruct tomographic image data of one slice, projection data for approximately 360° corresponding to one revolution around the object is necessary. A half scan method still requires projection data for 180°+α (α: fan angle). The present invention is applicable to both reconstruction methods. An example of the former method will be explained here. As a mechanism for converting incident X-rays into charges, an indirect conversion type that converts X-rays into light by a phosphor such as a scintillator and converts the light into charges by a photoelectric conversion element such as a photodiode, and a direct conversion type that uses electron-hole pair generation in a semiconductor by X-rays and their movement to the electrode, that is, photoconductive phenomenon are the mainstreams. As the X-ray detection element, either method is adoptable. In recent years, so-called multi-tube type X-ray computed tomography apparatuses in which a plurality of pairs of X-ray tubes and X-ray detectors are mounted in a rotating ring have been put on the market, and peripheral technologies have been developed. The present invention is applicable to either a conventional single-tube type X-ray computed tomography apparatus or the multi-tube type X-ray computed tomography apparatus. The single-tube type will be explained here.

To enable data collection in a desired cardiac phase while avoiding prolongation of the scanning time as much as possible even if an arrhythmia has occurred, the X-ray computed tomography apparatus according to this embodiment characteristically includes an arrhythmia index calculation unit and a mode selection unit. The arrhythmia index calculation unit calculates an arrhythmia index representing the degree of arrhythmia using the biological information of an object. The mode selection unit selects a normal imaging mode or an arrhythmia imaging mode in advance based on the arrhythmia index. In the normal imaging mode, after the elapse of a predetermined delay time from the characteristic wave of the heartbeat waveform of the object, projection data is collected along with X-ray generation. Upon detecting an arrhythmia, the X-ray generation is temporarily stopped. In the arrhythmia imaging mode, after the elapse of a predetermined delay time from the characteristic wave, projection data is collected along with X-ray generation. Upon detecting an arrhythmia, an arrhythmia scanning procedure corresponding to an arrhythmia type is temporarily executed, and projection data is collected along with X-ray generation.

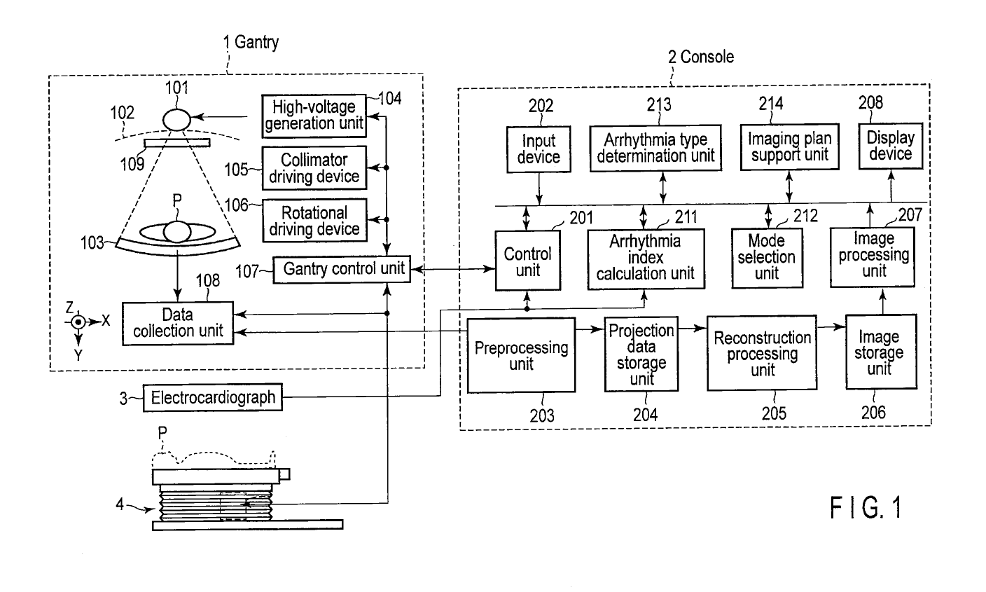

FIG. 1 shows the arrangement of an X-ray computed tomography apparatus according to this embodiment. The X-ray computed tomography apparatus includes a gantry 1 configured to collect projection data concerning an object. The gantry 1 includes an X-ray tube 1011 and an X-ray detector 103. The X-ray tube 101 and the X-ray detector 103 are mounted on a ring-shaped rotary frame 102 to be rotationally driven by a rotational driving device 106. For the descriptive convenience, the rotating central axis of the rotary frame 102 is defined as the z-axis, the x-axis is defined in the horizontal direction, and the y-axis is defined in the vertical direction. The rotary frame 102 has an opening at the center. An object P lied down on the top of a bed 4 is inserted into the opening. An electrocardiograph 3 is attached to the object P. The electrocardiograph 3 measures the electrical phenomenon of the heart of the object P, and outputs electrocardiogram data as the time-rate change. Note that the electrocardiograph 3 may typically detect an R wave peak as a characteristic wave from a cardiogram, and output a specific signal at that timing.

A high-voltage generation unit 104 applies a tube voltage between the cathode and the anode of the X-ray tube 101. The high-voltage generation unit 104 also supplies a filament current to the filament of the X-ray tube 101. X-rays are generated by applying the tube voltage and supplying the filament current. The radiation window of the X-ray tube 101 is provided with an X-ray collimator 109. The X-ray detector 103 includes a plurality of X-ray detection elements arrayed in the vertical and horizontal directions. A data collection unit 108 generally called DAS converts a signal output from the X-ray detector 103 for each channel into a voltage signal, amplifies it, and converts the signal into a digital signal. This data (raw data) is supplied to a console 2 outside the gantry. A gantry control unit 107 controls the high-voltage generation unit 104, a collimator driving device 105, and the rotational driving device 106 to execute an scanning procedure (scanning) instructed from a control unit 201 of the console 2.

A preprocessing unit 203 of the console 2 performs correction processing such as sensitivity correction for the data output from the data collection unit 108, thereby generating projection data. The projection data is stored in a projection data storage unit 204. A reconstruction processing unit 205 reconstructs 3D distribution data (to be simply referred to as volume data hereinafter) of CT values based on the projection data for, for example, 360°. The volume data is stored in an image storage unit 206. An image processing unit 207 performs MPR (Multi-Planar Reconstruction), volume rendering processing, and the like to generate, from the volume data, a 2D image displayable on the screen of a display device 208.

The console 2 includes an arrhythmia index calculation unit 211, a mode selection unit 212, an arrhythmia type determination unit 213, and an scanning plan support unit 214 as constituent elements characteristic in the embodiment. The arrhythmia index calculation unit 211 calculates an arrhythmia index (also referred to as an arrhythmia level hereinafter) representing the degree of variation in the heartbeat cycle of the object using the cardiogram of the object measured by the electrocardiograph 3. The arrhythmia index exhibits a relatively high value when the variation in the heartbeat cycle is relatively large. The arrhythmia index exhibits a relatively low value when the variation in the heartbeat cycle is relatively small. The heartbeat cycle is given as a time interval from a peak of an R wave that is a characteristic wave to the next peak of the R wave, and the reciprocal thereof indicates the heart rate. An arrhythmia index CV (%) is obtained by, for example,

CV (%)=(standard deviation of RR/average value of RR)×100

where RR is the heartbeat cycle. The heartbeat cycle concerning the nth beat is represented by RRn.

The mode selection unit 212 selects one of the normal imaging mode and the arrhythmia imaging mode based on the arrhythmia index calculated by the arrhythmia index calculation unit 211. When no arrhythmia occurs, the same scanning procedure (normal scanning procedure) is used in both the normal imaging mode and the arrhythmia imaging mode. For the normal scanning procedure, the average heartbeat cycle of the object is obtained in advance. After the elapse of a standby time corresponding to a cardiac phase desired for data collection, for example, a middiastolic phase from the R wave in the entire average heartbeat cycle, the object is irradiated with X-rays only during a predetermined period corresponding to the middiastolic phase, thereby executing projection data collection for the object.

Note that projection data collection by the data collection unit 108 via the X-ray detector 108 is repeated at a predetermined period independently of the presence/absence of X-ray generation (irradiation). Effective projection data collected under X-ray irradiation is selectively used for image reconstruction processing under the control of the control unit 201. Data output from the data collection unit 108 in an X-ray non-irradiation state is not selectively used for image reconstruction processing under the control of the control unit 201. For the descriptive convenience, the simple expression “projection data collection” is assumed to mean “collection of effective projection data under X-ray irradiation”.

If the variation duration (the absolute value of the difference) of the heartbeat cycle with respect the immediately preceding heartbeat cycle exceeds a predetermined threshold, the control unit 201 recognizes occurrence of an arrhythmia. In the arrhythmia imaging mode, when occurrence of an arrhythmia is recognized, an arrhythmia scanning procedure that is different from the normal scanning procedure and is selected in advance based on the arrhythmia type is temporarily executed in place of the normal scanning procedure. Details will be described later.

In the normal scanning procedure, when occurrence of an arrhythmia is recognized, X-ray generation is stopped at that point of time, and the normal scanning procedure is resumed from the current R wave or the next R wave. To actually temporarily stop the normal scanning procedure, X-ray generation is stopped along with the stop of the X-ray irradiation. Alternatively, even when X-ray generation is continued, the intensity of the X-rays is lowered by modulating the tube current. Typically, the X-ray stop is done by stopping tube current application, stopping filament current supply, or stopping discharge of thermoelectrons by grid control. The tube current modulation is done by filament current control or grid control. For the descriptive convenience, the actual temporary stop of the normal scanning procedure is assumed to be the stop of X-ray generation.

In the arrhythmia imaging mode, the arrhythmia scanning procedure is selected in advance based on the arrhythmia type before the start of scanning. The arrhythmia type determination unit 213 determines the arrhythmia type based on the classification result of each of a plurality of heartbeat cycles and the frequency distribution of the plurality of heartbeat cycles in the cardiogram measured on the object for one to several min in the scanning planning stage. As is known, extrasystole (compensatory), extrasystole (interpolated), and the like have been grasped as the arrhythmia types. Classifying each of the plurality of heartbeat cycles means classifying a heartbeat cycle based on its time length. More specifically, when the difference time obtained by subtracting the immediately preceding heartbeat cycle from the current heartbeat cycle exceeds a predetermined upper threshold, the heartbeat cycle is classified as a type “long”. When the difference time obtained by subtracting the immediately preceding heartbeat cycle from the current heartbeat cycle has a negative polarity and smaller than a predetermined lower threshold, the heartbeat cycle is classified as a type “short”. When the difference time obtained by subtracting the immediately preceding heartbeat cycle from the current heartbeat cycle falls within the range between the upper threshold and the lower threshold, the heartbeat cycle is classified as a type “standard”. Based on how the classification results are distributed in the frequency distribution, a most similar pattern is selected from a plurality of patterns prepared in advance for the plurality of arrhythmia types, thereby determining the arrhythmia type. Note that as a simple method, the arrhythmia type is determined based on the relationship between three heartbeat cycles, that is, a heartbeat cycle representing the maximum frequency out of the heartbeat cycles classified as “long”, a heartbeat cycle representing the maximum frequency out of the heartbeat cycles classified as “short”, and a heartbeat cycle representing the maximum frequency out of the heartbeat cycles classified as “standard”.

Note that the above-described arrhythmia may be detected based on the waveform feature of the cardiogram of the object. The waveform feature of the cardiogram can not only detect the arrhythmia but also specify the arrhythmia type. As is known, the waveform of a cardiogram is characterized by a plurality of parameters such as an RR interval (heartbeat cycle) from an R wave peak to the next R wave peak, a PQ interval from the leading edge of a P wave to a point immediately before a Q wave, a PQ interval from the leading edge of a P wave to a point immediately before a Q wave, a QRS width from the leading edge of a Q wave to the end of an S wave through an R wave, a QT interval from the leading edge of a Q wave to the end of a T wave, and the peak value of the R wave. In this embodiment, the arrhythmia detection and the arrhythmia type specification may be done based on all or some of the plurality of parameters.

When the arrhythmia type is determined, the scanning plan support unit 214 displays, on the display device 208, a setting screen that prompts the operator to do settings of detailed parameters of the arrhythmia scanning procedure via an input device 202 based on the type and select one scanning plan out of continuous irradiation, tube current modulation, and X-ray on/off for the arrhythmia procedure via the input device 202.

After selection of the normal imaging mode/arrhythmia imaging mode and the settings of the arrhythmia scanning procedure are determined as described above, scanning (data collection) is actually executed under the control of the control unit 201 in accordance with a trigger from the operator along with arrhythmia detection by the control unit 201.

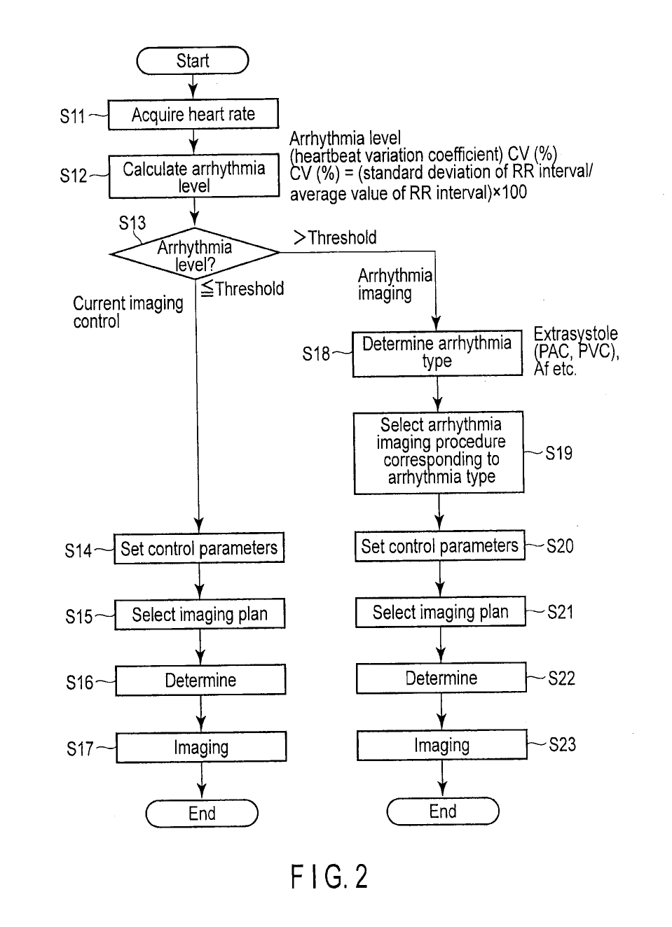

FIG. 2 illustrates a procedure up to completion of scanning mainly including the procedure of laying down an scanning plan according to the embodiment. Referring to FIG. 2, steps S11 to S16 and S18 to S22 indicate the steps in the scanning planning stage, and steps S17 and S22 indicate the scanning steps. To put an scanning plan, first, the electrocardiograph 3 measures the cardiogram of the object P for an arbitrarily settable predetermined period, for example, 60 sec under the control of the control unit 201. The control unit 201 acquires a heartbeat cycle (heart rate) for each heartbeat based on the R wave on the heartbeat waveform (step S11). The heartbeat cycle means the time interval from an R wave peak to the next R wave peak. Note that if the electrocardiograph 3 has a function of detecting an R wave and outputting a pulse signal, the control unit 201 acquires a heartbeat cycle for each heartbeat based on the pulse signal. A plurality of, for example, 70 heartbeat cycles are acquired.

The arrhythmia index calculation unit 211 calculates an arrhythmia index (arrhythmia level) based on the plurality of heartbeat cycles (step S12). As described above, the arrhythmia index CV (%) is given by the ratio of the standard deviation of the heartbeat cycle RR to the average value of the heartbeat cycle RR. Hence, an arrhythmia index exhibiting a relatively high value represents that the variation in the heartbeat cycle is relatively large, that is, the probability that an arrhythmia will occur is high. The mode selection unit 212 compares the arrhythmia index with a threshold (step S13). The threshold is set by the mode selection unit 212 based on the height, weight, gender, age, anamnesis, and the like of the object. However, the threshold can be changed to an arbitrary value in accordance with an operation instruction via the input device 202. If the arrhythmia index is equal to or smaller than the threshold, the normal imaging mode is selected. If the arrhythmia index exceeds the threshold, the arrhythmia imaging mode is selected.

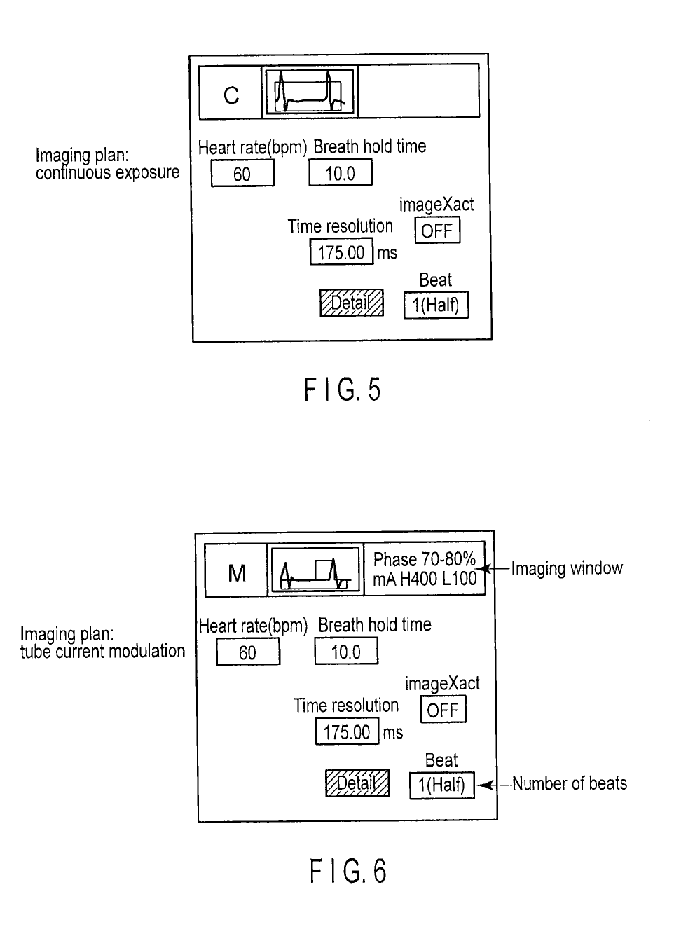

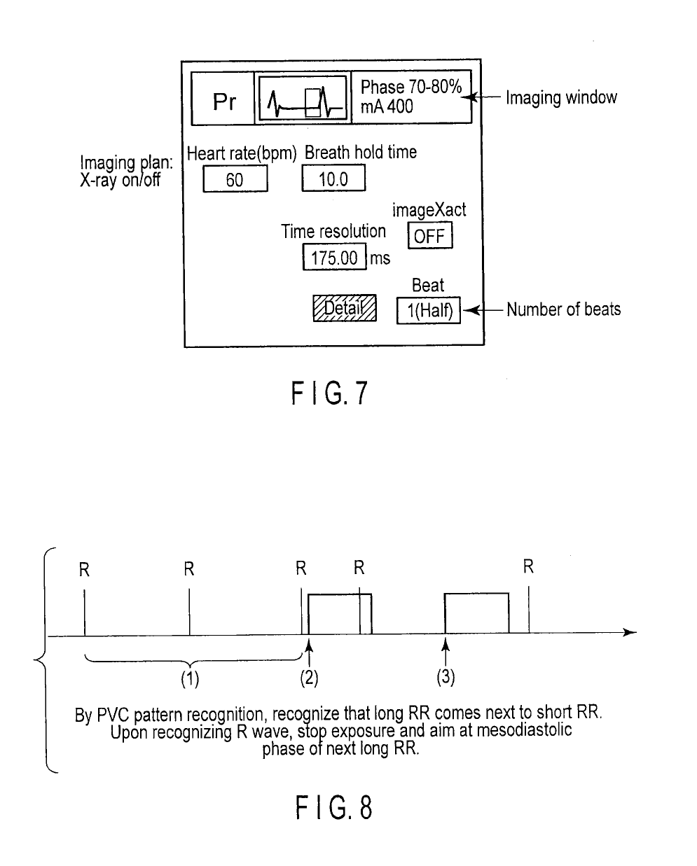

When the normal imaging mode is selected, the scanning plan support unit 214 displays, on the display device 208, a screen to input detailed scanning parameters in the normal imaging mode. FIGS. 5, 6, and 7 show examples of the screen. The screen includes the breath hold time, the measurement frequency (time resolution) of the heartbeat cycle (heart rate) during the scanning period, scanning stop/continuation (off/on) upon detecting an arrhythmia, and the heart rate (the number of scanning beats excluding the heart rate upon detecting an arrhythmia) necessary for obtaining projection data of one set (360° or (180°+fan angle)) required for image reconstruction. The operator can arbitrarily set these parameters (step S14). The operator can also select an scanning plan together with the scanning parameters (step S15). The scanning plan is selected arbitrarily from a continuous exposure plan for continuously emitting X-rays, a tube current modulation plan for controlling X-rays by modulating the tube current, and an X-ray on/off plan for turning on/off X-rays by opening/closing the shutter or grid control in accordance with an operator instruction, as shown in FIGS. 5, 6, and 7, as a condition for the technique of stopping X-ray generation.

When the scanning parameters and the scanning plan are determined (step S16), scanning (data collection and scanning) starts in response to an scanning trigger button operation by the operator.

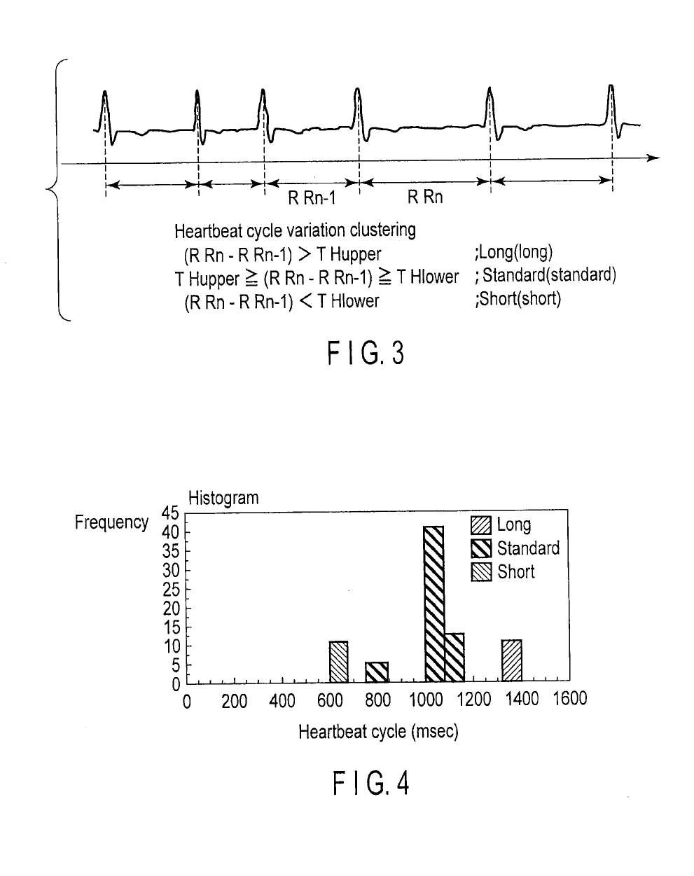

When the arrhythmia index exceeds the threshold, and the arrhythmia imaging mode is selected in step S13, the arrhythmia type determination unit 213 determines the arrhythmia type (step S18). The arrhythmia types include extrasystole (compensatory), extrasystole (interpolated), and the like, and one of them is specified. To determine the arrhythmia type, the arrhythmia type determination unit 213 classifies each of the plurality of heartbeat cycles and generates a frequency distribution for the plurality of heartbeat cycles. As shown in FIG. 3, the heartbeat cycles are classified into three types by the degree of change in the duration of each heartbeat cycle with respect to the immediately preceding heartbeat cycle and the difference in length. The immediately preceding heartbeat cycle RRn−1 is subtracted from each heartbeat cycle RRn. If the difference time (with a +/− polarity) exceeds a predetermined upper threshold THupper, the heartbeat cycle is much longer than the immediately preceding heartbeat cycle and is classified as the type “long”. If the difference time is less than a predetermined lower threshold THlower, the heartbeat cycle is much shorter than the immediately preceding heartbeat cycle and is classified as the type “short”. If the difference time falls within the range between the upper threshold (inclusive) and the lower threshold (inclusive), the heartbeat cycle does not largely vary from the immediately preceding heartbeat cycle and is classified as the type “standard”. The upper threshold and the lower threshold can individually be changed from the initial values based on the height, weight, gender, age, anamnesis, and the like of the object to arbitrary values in accordance with an operator's instruction via the input device 202.

As shown in FIG. 4, the appearance frequency of the plurality of heartbeat cycles is counted for each time zone, and the classification results are merged with the resultant frequency distribution. The internal memory of the arrhythmia type determination unit 213 stores typical frequency distributions corresponding to the plurality of arrhythmia types, which are prepared in advance, as pattern data together with the classification result of each heartbeat cycle. A pattern most similar to the frequency distribution and classification result for the object is selected from these patterns. The arrhythmia type is thus determined. As a simple determination method, the arrhythmia type is determined based on the relationship in the length of time between three heartbeat cycles, that is, a heartbeat cycle representing the maximum frequency out of the heartbeat cycles classified as “long”, a heartbeat cycle representing the maximum frequency out of the heartbeat cycles classified as “short”, and a heartbeat cycle representing the maximum frequency out of the heartbeat cycles classified as “standard”.

Upon determining the arrhythmia type, the scanning plan support unit 214 selects the arrhythmia scanning procedure corresponding to the arrhythmia type. The arrhythmia scanning procedure is an scanning procedure when the control unit 201 has detected an arrhythmia during the scanning period, and is different from the normal scanning procedure when no arrhythmia type is detected. An example of the arrhythmia scanning procedure preset in accordance with the arrhythmia type is as follows.

### (Extrasystole(Compensatory),

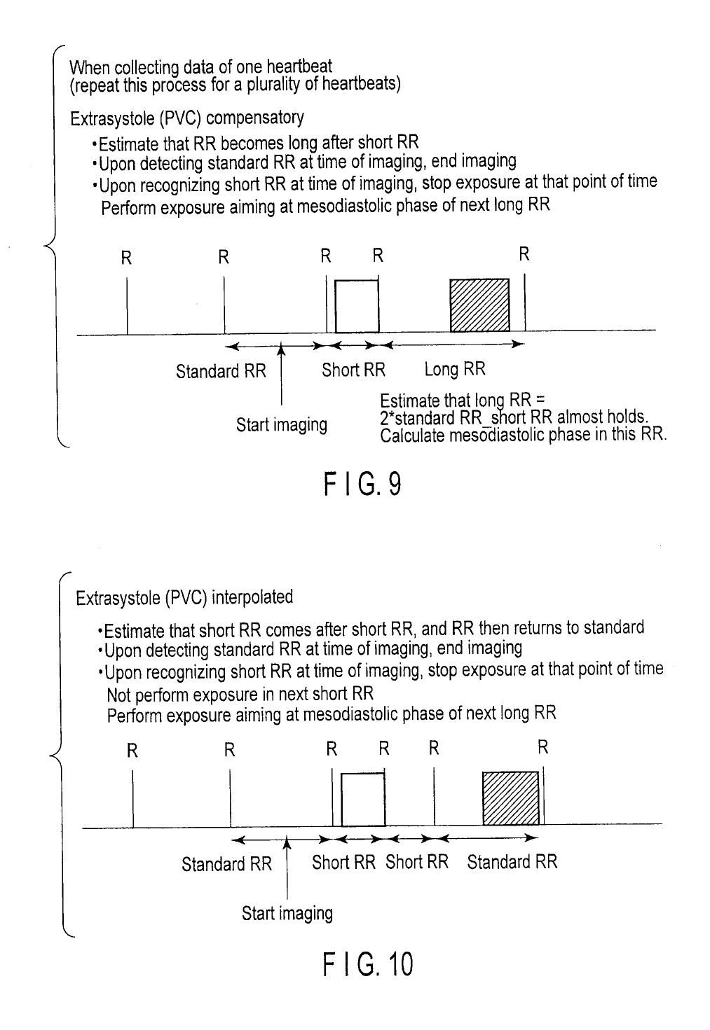

### Extrasystole(Interpolated), See FIGS. 8 and 9).

In an arrhythmia of this type, typically, a “short” heartbeat cycle arrives next to a “standard” heartbeat cycle, and then, a “long” heartbeat cycle arrives. In many cases, the desired cardiac phase is the middiastolic phase. Upon detecting a “short” heartbeat cycle (arrhythmia detection), scanning (data collection) is performed in the middiastolic phase of a “long” heartbeat cycle that occurs at a high probability next to the “short” heartbeat cycle in this arrhythmia type. The “long” heartbeat cycle can be estimated to have a time length obtained by subtracting the “short” heartbeat cycle from a time length twice the “standard” heartbeat cycle. A time range corresponding to the middiastolic phase is set as the scanning period for the estimated heartbeat cycle. That is, for the arrhythmia of compensatory extrasystole (PVC) type, it is estimated that a long heartbeat cycle arrives after a short heartbeat cycle. When the short heartbeat cycle is recognized at the time of scanning, X-ray exposure is stopped at that point of time. Then, scanning is performed in the middiastolic phase of the long heartbeat cycle.

### (Extrasystole(Compensatory),

### Extrasystole(Interpolated), See FIG. 10)

In an arrhythmia of this type, it can be estimated that a short heartbeat cycle arrives next to a short heartbeat cycle, and the heartbeat cycle then returns to the standard. When the short heartbeat cycle is detected at the time of scanning, X-ray exposure is stopped at that point of time. Then, the next short heartbeat cycle is skipped, and scanning is performed in the middiastolic phase of the next “long” heartbeat cycle.

### (Ventricular Fibrillation (Af))

In this type, the arrhythmia level is high, and the standard heartbeat cycle occurs at random. Note that if the heartbeat cycle is much longer than the standard heartbeat cycle, and collection of projection data of one set can be completed, scanning ends in one heartbeat. When the heartbeat of such a long heartbeat cycle has not arrived during an arbitrary standby period from the start of scanning, for example, until three heartbeat periods have elapsed, projection data collected during two heartbeats (the number of scanning beats is 2) between the fourth heartbeat and the fifth heartbeat of the standard heartbeat cycle is applied to image reconstruction. The standby period and the number of scanning beats can arbitrarily be set.

Note that in steps S20 and S21, the parameters such as the breath hold time, scanning stop/continuation (off/on) upon detecting an arrhythmia, and the number of scanning beats are set, and the scanning plan is selected, as in steps S14 and S15. As for the parameter setting, the scanning plan support unit 214 provides set values corresponding to the arrhythmia level and the arrhythmia type. For example, the heart rate (the number of scanning beats) for which the scanning is repeated in accordance with the arrhythmia scanning procedure upon detecting an arrhythmia is set to 2 or 3 heartbeats. When the scanning plan is tube current modulation or X-ray on/off, supports are made by, for example, prolonging the scanning period as the arrhythmia level rises or automatically selecting continuous exposure when the arrhythmia level exhibits an excessively high value.

After selection of the arrhythmia imaging mode and determination of the arrhythmia scanning procedure (step S21), scanning (data collection) is actually executed under the control of the control unit 201 in accordance with a trigger from the operator (step S22).

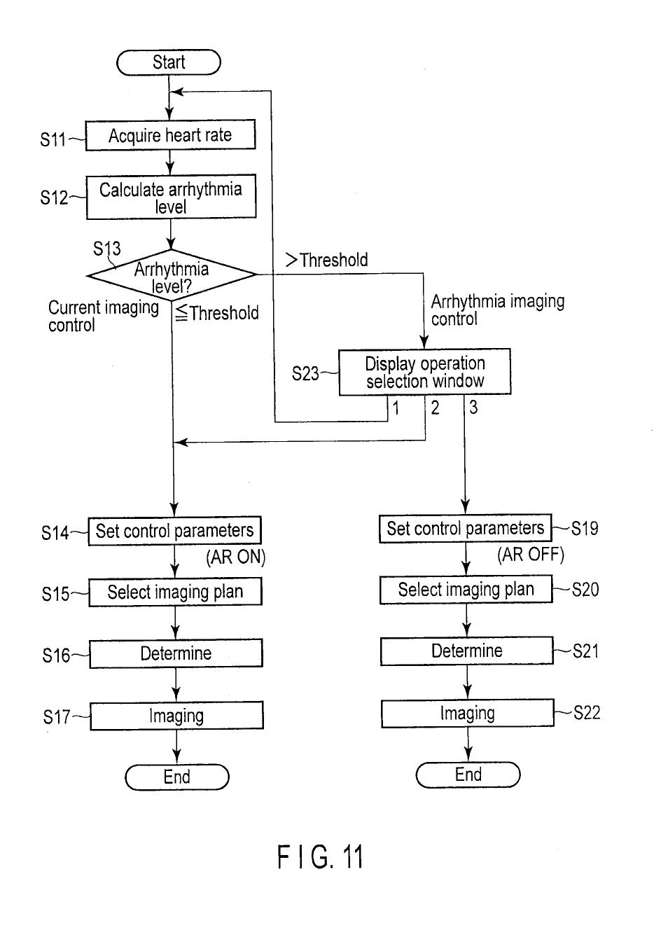

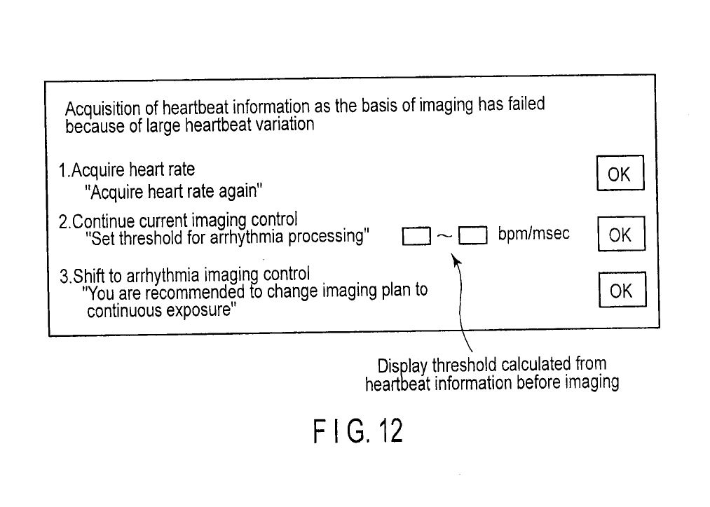

Note that even if the arrhythmia level exceeds the threshold in step S13, the scanning plan support unit 214 may display a screen to prompt the operator to select one of (1) reacquisition of the heart rate, (2) designation of the normal imaging mode along with manually setting the threshold for arrhythmia detection, and (3) designation of the arrhythmia imaging mode, as shown in FIGS. 11 and 12 (step S23).

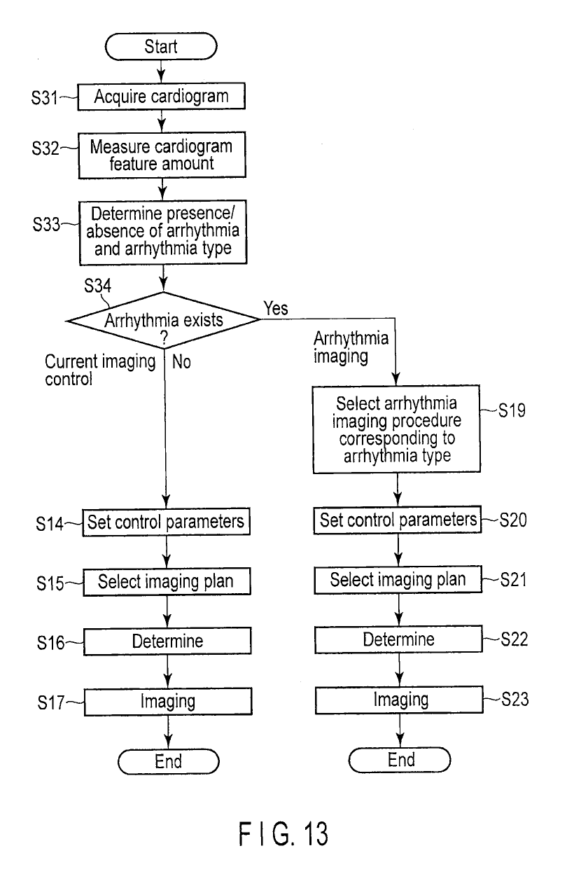

FIG. 13 illustrates a processing procedure of performing arrhythmia detection and arrhythmia type specification using a cardiogram. The same step numbers as in FIG. 2 denote the same steps in FIG. 13. As a preparation, a cardiogram is acquired for one to several min using the electrocardiograph 3 (step S31). The arrhythmia type determination unit 213 measures a plurality of parameters representing the waveform feature from the waveform of the cardiogram for each heartbeat cycle (step S32). For example, the arrhythmia type determination unit 213 measures a plurality of parameters such as an RR interval (heartbeat cycle) from an R wave peak to the next R wave peak, a PQ interval from the leading edge of a P wave to a point immediately before a Q wave, a PQ interval from the leading edge of a P wave to a point immediately before a Q wave, a QRS width from the leading edge of a Q wave to the end of an S wave through an R wave, a QT interval from the leading edge of a Q wave to the end of a T wave, and the peak value of the R wave. The arrhythmia type determination unit 213 stores the correspondence table of arrhythmia types for combinations of the plurality of parameters in the internal ROM or an external ROM. The arrhythmia type determination unit 213 inquires of the arrhythmia type correspondence table about the plurality of measured parameters, thereby specifying the presence/absence of an arrhythmia and the arrhythmia type (step S33).

If no arrhythmia is detected (no arrhythmia exists), the mode selection unit 212 executes the process in step S14. Upon detecting an arrhythmia (an arrhythmia exists), the mode selection unit 212 executes the process in step S19. Since the arrhythmia type is specified from the plurality of parameters in step S33, step S18 of FIG. 2 is unnecessary. The subsequent processing is the same as that of FIG. 2.

In the above description, the heartbeat cycle is obtained from a cardiogram or an R wave signal thereof. However the heartbeat cycle may be obtained from pulse waves or heart sound.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography apparatus comprising:
an X-ray tube;
a high-voltage generation unit configured to generate a high voltage to be applied between electrodes of the X-ray tube to generate X-rays from the X-ray tube;
an X-ray detector configured to detect the X-rays generated by the X-ray tube and passed through an object;
a data collection unit configured to collect projection data concerning the object via the X-ray detector;
an arrhythmia index calculation unit configured to calculate an arrhythmia index representing a degree of arrhythmia concerning the object using biological information of the object;
a mode selection unit configured to select one of a normal imaging mode and an arrhythmia imaging mode in advance based on the arrhythmia index, the normal imaging mode collecting the projection data along with X-ray generation after an elapse of a predetermined delay time from a characteristic wave of a heartbeat waveform of the object, and upon detecting an arrhythmia, temporarily stopping the X-ray generation, and the arrhythmia imaging mode collecting the projection data along with the X-ray generation after the elapse of the predetermined delay time from the characteristic wave, and upon detecting the arrhythmia, temporarily executing an arrhythmia scanning procedure corresponding to an arrhythmia type and collecting the projection data along with the X-ray generation; and
a control unit configured to control the high-voltage generation unit and the data collection unit to collect the projection data in accordance with the selected one of the normal imaging mode and the arrhythmia imaging mode.

2. The X-ray computed tomography apparatus according to claim 1, further comprising an arrhythmia type specification unit configured to specify the arrhythmia type based on a frequency distribution of heartbeat cycles of the object.

3. The X-ray computed tomography apparatus according to claim 2, wherein the arrhythmia type specification unit classifies the heartbeat cycles into at least three types in accordance with one of a variation time and a variation ratio to a heartbeat cycle of an immediately preceding heartbeat.

4. The X-ray computed tomography apparatus according to claim 3, wherein the arrhythmia type specification unit specifies the arrhythmia type based on a pattern of the frequency distribution discriminated by the type.

5. The X-ray computed tomography apparatus according to claim 4, wherein the arrhythmia type specification unit specifies the arrhythmia type by selecting a standard pattern most approximate to the pattern of the frequency distribution discriminated by the type from a plurality of standard patterns defined for the respective arrhythmia types.

6. The X-ray computed tomography apparatus according to claim 1, further comprising an arrhythmia type specification unit configured to specify the arrhythmia type based on a cardiogram of the object.

7. The X-ray computed tomography apparatus according to claim 6, wherein the arrhythmia type specification unit specifies the arrhythmia type based on a plurality of parameters including a QRS width on the cardiogram of the object.

8. The X-ray computed tomography apparatus according to claim 1, wherein the arrhythmia index calculation unit calculates the arrhythmia index in accordance with a ratio of a standard deviation of the heartbeat cycle to an average value of the heartbeat cycle.

9. The X-ray computed tomography apparatus according to claim 1, wherein when the arrhythmia type is Extrasystole(Compensatory), Extrasystole(Interpolated), the X-ray generation is stopped, the next heartbeat cycle is skipped, and the X-ray generation is resumed assuming that the next heartbeat cycle is a relatively long cycle.

10. The X-ray computed tomography apparatus according to claim 1, wherein when the arrhythmia type is Extrasystole(Compensatory), Extrasystole(Interpolated), the X-ray generation is stopped, and the X-ray generation is resumed assuming that the next heartbeat cycle is a relatively long cycle.

11. An X-ray computed tomography apparatus comprising:
an X-ray tube;
a high-voltage generation unit configured to generate a high voltage to be applied between electrodes of the X-ray tube to generate X-rays from the X-ray tube;
an X-ray detector configured to detect the X-rays generated by the X-ray tube and passed through an object;
a data collection unit configured to collect projection data concerning the object via the X-ray detector; and
a control unit configured to control the high-voltage generation unit and the data collection unit to collect the projection data along with X-ray generation after an elapse of a predetermined delay time from a characteristic wave of a heartbeat waveform of the object, and upon detecting an arrhythmia, temporarily execute an arrhythmia scanning procedure corresponding to an arrhythmia type and collect the projection data along with the X-ray generation.

12. The X-ray computed tomography apparatus according to claim 11, further comprising an arrhythmia type specification unit configured to specify the arrhythmia type based on a frequency distribution of heartbeat cycles of the object.

13. The X-ray computed tomography apparatus according to claim 12, wherein the arrhythmia type specification unit classifies the heartbeat cycles into at least three types in accordance with one of a variation time and a variation ratio to a heartbeat cycle of an immediately preceding heartbeat.

14. The X-ray computed tomography apparatus according to claim 13, wherein the arrhythmia type specification unit specifies the arrhythmia type based on a pattern of the frequency distribution discriminated by the type.

15. The X-ray computed tomography apparatus according to claim 14, wherein the arrhythmia type specification unit specifies the arrhythmia type by selecting a standard pattern most approximate to the pattern of the frequency distribution discriminated by the type from a plurality of standard patterns defined for the respective arrhythmia types.

16. The X-ray computed tomography apparatus according to claim 11, further comprising an arrhythmia type specification unit configured to specify the arrhythmia type based on a cardiogram of the object.

17. The X-ray computed tomography apparatus according to claim 16, wherein the arrhythmia type specification unit specifies the arrhythmia type based on a plurality of parameters including a QRS width included in the cardiogram of the object.

18. The X-ray computed tomography apparatus according to claim 11, wherein the arrhythmia index calculation unit calculates the arrhythmia index in accordance with a ratio of a standard deviation of the heartbeat cycle to an average value of the heartbeat cycle.

19. An X-ray computed tomography apparatus comprising:
an X-ray tube;
a high-voltage generation unit configured to generate a high voltage to be applied between electrodes of the X-ray tube to generate X-rays from the X-ray tube;
an X-ray detector configured to detect the X-rays generated by the X-ray tube and passed through an object;
a data collection unit configured to collect projection data concerning the object via the X-ray detector;
an arrhythmia index calculation unit configured to calculate an arrhythmia index representing a degree of arrhythmia concerning the object using biological information of the object; and
a control unit configured to control the high-voltage generation unit and the data collection unit to change an scanning procedure of ECG-gated scan in accordance with the arrhythmia index.

20. An X-ray computed tomography apparatus comprising:
an X-ray tube;
a high-voltage generation unit configured to generate a high voltage to be applied between electrodes of the X-ray tube to generate X-rays from the X-ray tube;
an X-ray detector configured to detect the X-rays generated by the X-ray tube and passed through an object;
a data collection unit configured to collect projection data concerning the object via the X-ray detector;
an arrhythmia index calculation unit configured to calculate an arrhythmia index representing a degree of arrhythmia concerning the object using biological information of the object;
an scanning plan support unit configured to, if the arrhythmia index is higher than a predetermined threshold, display a screen to prompt an operator to input an instruction to select one of a normal imaging mode in which the projection data is collected along with X-ray generation during an scanning period of a predetermined duration after an elapse of a predetermined delay time from a characteristic wave of a heartbeat waveform of the object, and upon detecting an arrhythmia, the X-ray generation is temporarily stopped, and an arrhythmia imaging mode in which the projection data is collected along with the X-ray generation after the elapse of the predetermined delay time from the characteristic wave, and upon detecting the arrhythmia, an arrhythmia scanning procedure corresponding to an arrhythmia type is temporarily executed, and the projection data is collected along with the X-ray generation;
a mode selection unit configured to select the normal imaging mode when the arrhythmia index is lower than the predetermined threshold, and select one of the normal imaging mode and the arrhythmia imaging mode instructed to select by the operator when the arrhythmia index is higher than the predetermined threshold; and
a control unit configured to control the high-voltage generation unit and the data collection unit to collect the projection data in accordance with the selected one of the normal imaging mode and the arrhythmia imaging mode.
